(19) United States
(12) Patent Application Publication
(10) Pub. No.: US 2010/0274253 A1
(43) Pub. Date: Oct. 28, 2010

(54) Device and method for achieving accurate positioning of acetabular cup during total hip replacement

(76) Inventor: Keith J. Ure, Mt. Shasta City, CA (US)

Correspondence Address: MICHAEL J. URE, 10518 PHIL PLACE, CUPERTINO, CA, 95014, US

(21) Appl. No.: 12/429,167

(22) Filed: Apr. 23, 2009

(51) Int. Cl. A61B 17/58; A61F 5/00

(52) U.S. Cl. 606/91; 606/87

(57) ABSTRACT

A method and device are provided in order to achieve optimal or desired orientation of an acetabular cup for total hip replacement or hip resurfacing. The method and device utilize preoperative medical imaging such as CT or MRI scans, 3D computer modeling and a patient-specific alignment jig created from medical imaging data such as CT or MRI data and computer 3D modeling. The device allows accurate placement of a drill hole to establish an acetabular axis, and placement of an acetabular cup perpendicular to the axis.

### FIELD OF THE INVENTION

The present invention relates to joint implants, particularly hip joint implants.

### BACKGROUND OF THE INVENTION

Accurate acetabular cup position within the bony acetabulum is critical for successful function of a total hip prosthesis. Malposition of the cup can cause instability of the total hip implants, resulting in dislocation of the femoral head from within the cup. Malposition of the cup is also responsible for premature wear of the bearing surfaces. Both these events are a common cause for repeat surgery requiring revision of one or all of the components of a total hip replacement.

Ideal cup position is described by an inclination angle, and an anteversion angle. Both are important in achieving ideal cup position. Prior art efforts for accurate acetabular cup positioning have included an alignment post on the device used to insert the acetabular cup. The alignment post typically is oriented at a 45 degree angle to the cup. Orientation of the alignment post vertically, in theory, would result in the cup being inserted into the acetabulum at a 45 degree inclination angle. However, multiple variables, such as patient position on the operating table, pelvic tilt, or poor estimation of whether the alignment post is truly vertical can compromise accurate cup position.

Anteversion angle is typically achieved by a longitudinal rod attached to the cup insertion device. Anteversion is achieved by rotating the longitudinal rod about the transverse axis. Visual estimation of the cup position relative to the position of the acetabulum is also used as a guide. Such visual estimation is difficult due to multiple variables.

Computer assisted navigation has been utilized in an effort to achieve more accurate cup position. Such technology typically utilizes a computer, a tracking system to monitor position and points of the acetabulum and pelvis, and computer software which determines implant orientation relative to the tracking points of the acetabulum and pelvis.

### Overview

A device and method is provided whereby accurate cup position within the acetabulum, including both inclination and anteversion, is achieved by means of medical imaging such as computerized tomography scans (CT scans) or magnetic resonance imaging (MRI) scans of the acetabulum obtained preoperatively, which are then used to create an alignment jig placed within the acetabulum at the time of surgery.

The alignment jig is patient-specific, and is created prior to surgery based on the data obtained from measurements and points of the patient's acetabulum and pelvis obtained from preoperative medical imaging such as CT or MRI scan. The medical imaging such as CT or MRI scans can accurately determine the diameter of the acetabulum and points on the acetabulum and pelvis for reference in order to create a patient-specific jig. Data obtained from medical imaging such as CT or MRI is inputted into 3D computer-aided-design software, after which computer-guided machinery such as a computer-guided laser etcher, computer-controlled stereolithography machine, or computer-guided lathe creates the disposable jig by means of existing CAD technologies.

The patient-specific jig, placed within the acetabulum, has multiple contact points on the margin and body of the jig which rest on the acetabulum or pelvis so as to achieve a predetermined position on the acetabulum of the jig with respect to an X, Y and Z axis, and three planes in space relative to the axes. Such planes are typically referred to as sagittal, axial, and coronal planes.

The patient-specific jig has a site for placing a drill hole within the dome of the patient's acetabulum. The orientation of the hole is determined from preoperative medical imaging such as CT or MRI scans, which provide images of the acetabulum in axial, coronal and sagittal planes. Utilizing patient-specific data from medical images such as CT or MRI images, the orientation of the drill hole of the jig placed within the acetabulum is made so as to create an axis of rotation for placement of the acetabular cup, relative to the drill hole. The drill hole then establishes the acetabular axis, which axis may then serve to orient the implant cup. The axis may pass through the center of the implant socket and may be substantially perpendicular to the plane of the face of the acetabular cup.

The acetabular cup is created so as to have a post, spike, or protruding element at the dome of the cup, which then is guided within the hole created by the patient-specific jig. By orienting the post to the drill hole placed within the acetabulum, precise cup position is created with respect to both inclination and anteversion. Exact inclination and anteversion can be based on typical parameters (usually between 40 and 45 degrees of inclination and between 15 and 20 degrees of anteversion), or specific inclination and anteversion angles unique to that particular patient, as determined by preoperative planning based on medical imaging such as CT or MRI evaluation.

Additional features and benefits of the present invention will become apparent from the detailed description, figures and claims set forth below.

### BRIEF DESCRIPTION OF THE DRAWINGS

The present invention will be understood more fully from the detailed description given below, and from the accompanying drawings of various embodiments of the invention, which, however, should not be taken to limit the invention to the specific embodiments, but are for explanation and understanding only.

FIG. 1 is a perspective view of a hemipelvis and drill guide for creation of a drill hole which establishes the acetabular axis.

FIG. 2 is a top perspective view of the acetabulum and drill guide.

FIG. 3 is a diagram illustrating a method of producing the drill guide.

### DETAILED DESCRIPTION

Means for establishing precise orientation and fixation of an acetabular cup for total hip replacement are discussed.

Those of ordinary skill in the art will realize that the following detained description of the present invention is illustrative only, and is not intended to be in any way limiting. Other embodiments of the present invention will readily suggest themselves to such skilled persons having the benefit of this disclosure. It will be apparent to one skilled in the art that these specific details may not be required to practice present invention. In other instances, well-known devices are shown in block diagram form to avoid obscuring the present invention. In the following description of the embodiments, substantially the same parts are denoted by the same reference numerals.

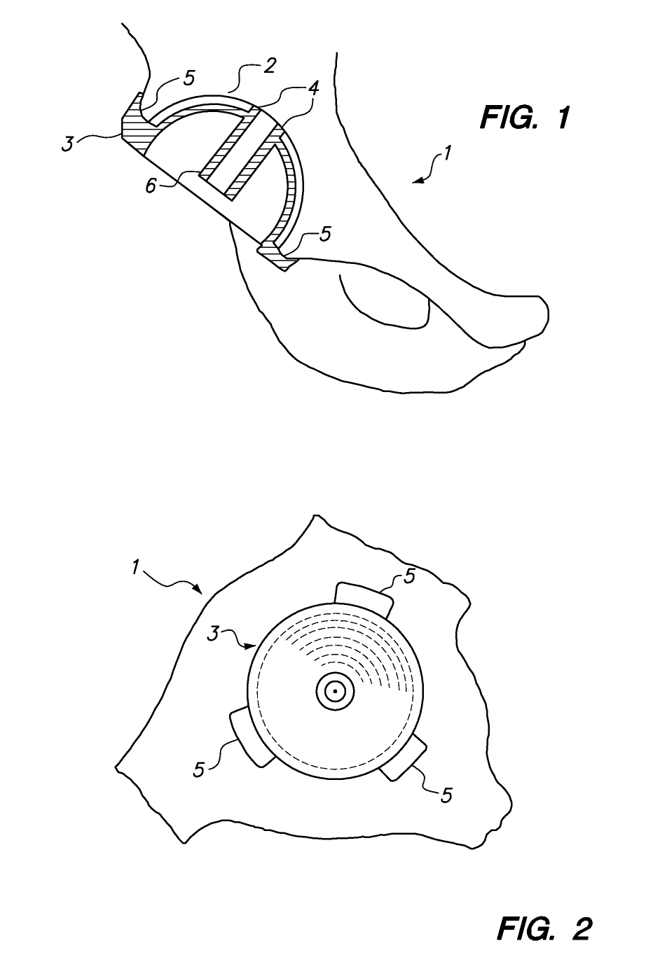

Referring now to FIG. 1, a perspective view is shown of a hemipelvis 1 and acetabulum 2, as well as a drill guide 3 for establishing the axis of the acetabular implant. The acetabulum 2 may have been prepared by reaming with hemispherical reamers to have created a hemispherical shape. The optimal or desired depth of the reaming can be established from preoperative medical imaging (e.g., CT or MRI scan) of the patient's pelvis, which will reveal the optimal or desired depth of reaming the acetabulum, relative to the inner wall of the pelvis, so as to avoid removing of excessive bone and possibly penetrating the inner wall of the pelvis. This optimal or desired depth may then be used to determine the distance between a base 4 of a drill bit sleeve 6 and outer contact points 5 of the drill guide 3 which rest on the outer wall of the acetabulum or pelvis. When optimal or desired reaming depth is achieved, the contact points 5 rest firmly on the outer walls of the acetabulum. If optimal or desired depth has not been achieved, the base 4 of the drill bit sleeve 6 causes the contact points 5 to be “proud” relative to the acetabular wall, i.e., not fully seated.

The drill bit sleeve 6 of the drill guide orients a drill bit so as to achieve optimal or desired orientation of the acetabular axis for the acetabular implant. The orientation of the drill bit sleeve 6 of the drill guide 3 will have been determined from preoperative medical imaging (e.g., CT or MRI scans) of the patient's pelvis with respect to anteversion and inclination and allow determination of the ideal or desired acetabular axis in order to establish the optimal or desired position of the face of the acetabular cup relative to the axis.

The acetabular cup may be of a generally hemispherical design, and may have a protrusion, post or spike which will then be guided into the hole drilled into the acetabulum with the drill guide 3.

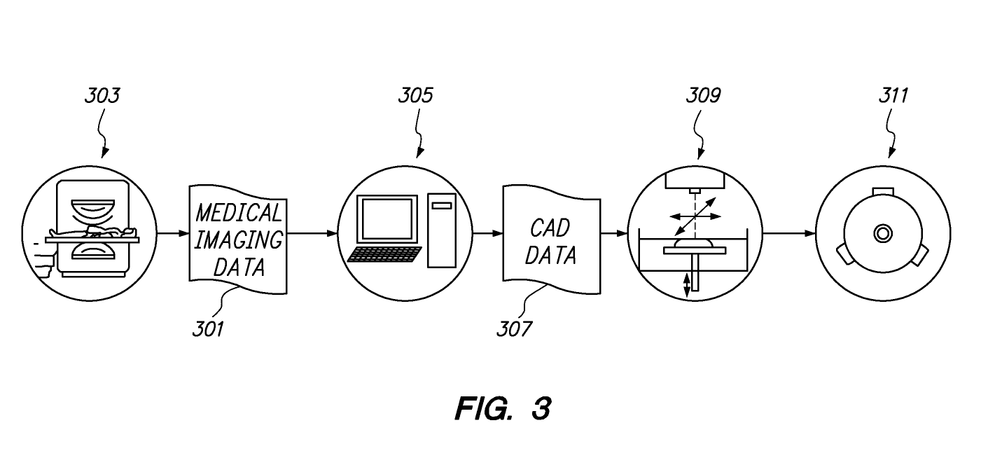

The alignment jig is patient-specific, and is created prior to surgery based on data obtained from measurements and points of the patient's acetabulum and pelvis obtained from preoperative medical imaging such as CT or MRI scan. Referring to FIG. 3, medical imaging such CT or MRI scans can accurately determine the diameter of the acetabulum and points on the acetabulum and pelvis for reference in order to create a patient-specific jig. Medical imaging data 301 obtained from a medical imaging apparatus 303 is inputted into 3D computer-aided-design software installed on a computer or workstation 305. The computer or workstation 305 produces CAD data 307 for the patient-specific jig, which is used to control computer-guided machinery such as a computer-guided laser etcher, computer-controlled stereolithography machine (illustrated in the present example), or computer-guided lathe 309 to create a disposable jig 311 by means of existing CAD technologies.

An advantageous cup design described in U.S. patent application Ser. No. 12/271,815 of the present inventor (Acetubular cup with supplemental screw fixation using conical interference fit between screw and cup, filed Nov. 14, 2008, incorporated herein by reference) takes advantage of the acetabular axis drill hole and achieve precise orientation of the acetabular cup relative to the sagittal, axial and coronal planes of the patients pelvis.

While particular embodiments of the present invention have been shown and described, it will be obvious to those skilled in the art that, based on the teachings herein, changes and modifications may be made without departing from this invention and its broader aspects. Therefore, the appended claims are intended to encompass within their scope all such changes and modifications as are within the true spirit and scope of this invention.

What is claimed is:

1. A method of controlling orientation of an acetabular implant by means of a patient-specific acetabular alignment jig, which allows for creation of a drill hole in the acetabulum, thereby establishing an acetabular axis.

2. The method of claim 1, comprising:
obtaining a medical imaging scan of a patient's pelvis;
mapping computer data from the medical imaging scan with respect to at least one of acetabular depth, diameter, outer wall morphology, inclination, anteversion, and desired acetabular axis; and
supplying the computer data to three-dimensional computer software.

3. The method of claim 2, comprising utilizing the computer data to create said patient-specific acetabular alignment jig using computer-guided machinery.

4. The method of claim 2, comprising mapping computer data from the medical imaging scan with respect to at least two of acetabular depth, diameter, outer wall morphology, inclination, anteversion, and desired acetabular axis.

5. The method of claim 2, comprising mapping computer data from the medical imaging scan with respect to at least three of acetabular depth, diameter, outer wall morphology, inclination, anteversion, and desired acetabular axis.

6. A method comprising:
affixing a patient-specific jig to an outer wall of a patient's acetabulum; and
drilling an acetabular axis hole in a base of the acetabulum so as to create a desired acetabular axis, in order to achieve accurate inclination and anteversion.

7. The method of claim 6, comprising placing an acetabular cup having a face and a projection substantially perpendicular to the face by inserting the projection into the acetabular axis hole, so as to achieve desired orientation of the face of the acetabular cup.

8. A patient-specific acetabular alignment jig produced by a method comprising:
obtaining a medical imaging scan of a patient's pelvis;
mapping computer data from the medical imaging scan with respect to at least one of acetabular depth, diameter, outer wall morphology, inclination, anteversion, and desired acetabular axis;
supplying the computer data to three-dimensional computer software; and utilizing the computer data to create a patient-specific alignment jig using computer-guided machinery.

9. The patient-specific acetabular alignment jig of claim 8, the method comprising mapping computer data from the medical imaging scan with respect to at least two of acetabular depth, diameter, outer wall morphology, inclination, anteversion, and desired acetabular axis.

10. The patient-specific acetabular alignment jig of claim 8, the method comprising mapping computer data from the medical imaging scan with respect to at least three of acetabular depth, diameter, outer wall morphology, inclination, anteversion, and desired acetabular axis.
